(19) United States
(12) Patent Application Publication
(10) Pub. No.: US 2008/0215136 A1
(43) Pub. Date: Sep. 4, 2008

(54) DIFFERENTIAL DRUG RELEASE FROM A MEDICAL DEVICE

(76) Inventors: Daniel J. Gregorich, St. Louis Park, MN (US); Mike Meyer, Richfield, MN (US)

Correspondence Address: JONES DAY, 222 EAST 41ST ST, NEW YORK, NY, 10017, US

(21) Appl. No.: 11/953,969

(22) Filed: Dec. 11, 2007

Related U.S. Application Data
(60) Provisional application No. 60/877,266, filed on Dec. 26, 2006.

(51) Int. Cl. A61F 2/82; A61F 2/06

(52) U.S. Cl. 623/1.39; 623/1.42; 623/1.43

(57) ABSTRACT

The invention relates to a medical device, such as an intravascular stent, useful for delivering two or more therapeutic agents to a body tissue of a patient at different rates, and methods for making and using such medical device. The medical device includes a substrate and/or coating having a plurality of pores, dispersed in said pores are a plurality of a first and second therapeutic agents, wherein said first therapeutic agent is bonded to one or more molecule(s) of a first material and the second therapeutic agent bonded to one or more molecule(s) of a second material, such that when the medical device, is in use (e.g., implanted into a body lumen such as a blood vessel), bonded first therapeutic agent is released from the medical device at a rate that is slower than the rate at which the bonded second therapeutic agent is released from the medical device.

### 1. FIELD OF THE INVENTION

The invention relates generally to medical devices that are useful for delivering, at different rates, two or more therapeutic agents to a body tissue, such as a vessel lumen. In particular, the invention is directed to an implantable medical device, preferably an intravascular stent, that releases different therapeutic agents, at different rates. More particularly, the invention is directed to an implantable or insertable medical device comprising a porous substrate and/or coating composition comprising a first therapeutic agent bonded to one or more molecule(s) of a first material to form a bonded first therapeutic agent and a second therapeutic agent bonded to one or more molecule(s) of a second material to form a bonded second therapeutic agent, wherein the bonded first therapeutic agent is greater in size (e.g., average diameter, volume), mass and/or nature than the bonded second therapeutic agent and the bonded second therapeutic agent is released from the medical device at a faster rate and/or greater amount than the bonded first therapeutic agent. Methods of making and using the medical device of the present invention are also provided.

### 2. BACKGROUND OF THE INVENTION

Different types of endoprostheses, including vascular grafts and graft-stent combinations can be provided with bio-active agents and used for minimally invasive procedures in body conduits. These endoprostheses are designed to perform specific function(s). In the case of stents, for example, as well as their use in vascular procedures, stents are used for treating cancerous blockages inside body passageways (e.g., esophagus, bile ducts, trachea, intestine, vasculature and urethra, among others) by holding open passageways which have been blocked by the cancerous growth or tumors.

For vascular procedures, a stent in the form of a wire mesh tube props open an artery that has recently been cleared using angioplasty. Usually, the stent stays in the artery permanently, holds it open, improves blood flow to the heart muscle and relieves symptoms. The stent procedure is fairly common, and various types of stents have been developed and used.

To reduce the possibility of restenosis and to locally deliver a biologically active material to a patient's lumen, various types of biologically active material-coated stents have been proposed. For example, U.S. Pat. No. 6,258,121 to Yang et al. discloses a stent having a polymeric coating for controllably releasing an included active agent such as paclitaxel, to inhibit restenosis following angioplasty.

However, there is a need for controlling the release rate of therapeutic agents from medical devices into the surrounding body tissue. If a therapeutic agent is released to the body tissue too quickly, the effect of the therapeutic agent may be greater or more sudden than desired. Conversely, if a therapeutic agent is released to the body tissue too slowly, the effect of the therapeutic agent may be lost or slower than desired. Moreover, if the delivery of more than one therapeutic agent is required, different release rates of the therapeutic agents may be desired because, for example, the immediate release of a therapeutic agent over a short time period to treat, manage or ameliorate one condition may be required, whereas, the release of a therapeutic agent over a prolonged period of time may be required for treating, managing or ameliorating another condition.

Accordingly, there is a need for controlling the release of more than one therapeutic agent from an implantable medical device to a targeted body tissue. In particular, there is a need for an implantable medical device capable of delivering more than one therapeutic agent from the same medical device at separate release rates.

### 3. SUMMARY OF THE INVENTION

To achieve the aforementioned objectives, the inventors have invented implantable or insertable drug-releasing medical devices comprising two or more therapeutic agents, wherein the release rates of the therapeutic agents differ. For example, while a first therapeutic agent is bonded to one or more molecule(s) of a first material to form a bonded first therapeutic agent, a second therapeutic agent is bonded to one or more molecule(s) of a second material to form a bonded second therapeutic agent, wherein the average diameter of the bonded first therapeutic agent is greater than the average diameter of the bonded second therapeutic agent

In certain embodiments, the invention relates to a medical device comprising a substrate comprising bonded first therapeutic agents and bonded second therapeutic agents, wherein when the medical device is in use (e.g., implanted into a body lumen such as a blood vessel), the bonded first therapeutic agent is released from the medical device at a first rate and the bonded second therapeutic agent is released from the medical device at a second rate that is faster than said first rate.

In certain embodiments, the invention relates to a medical device comprising a substrate, and a coating composition disposed on at least a portion of the substrate, wherein the coating composition comprises bonded first therapeutic agents and bonded second therapeutic agents, wherein when the medical device is in use (e.g., implanted into a body lumen such as a blood vessel), the bonded first therapeutic agent is released from the medical device at a first rate and the bonded second therapeutic agent is released from the medical device at a second rate that is faster than said first rate.

In specific embodiments, the second rate is about ten times, nine times, eight times, seven times, six times, five times, four times, three times, or two times faster than the first rate.

In one embodiment, the substrate is porous. In another embodiment, the coating composition is porous. In another embodiment, both the substrate and the coating composition are porous.

In certain embodiments, the average diameter of the pores in the substrate and/or coating composition is within a range of about 0.01 microns (μm) to about 200 μm, about 0.1 μm to about 180 μm, about 0.5 μm to about 160 μm, about 1 μm to about 140 μm, about 10 μm to about 120 μm, about 20 μm to about 100 μm, about 30 μm to about 80 μm, or about 40 μm to about 60 μm. In certain embodiments, the average diameter of the pores in the substrate and/or coating composition is about 0.01 μm, about 0.1 μm, about 1 μm, about 10 μm, about 20 μm, about 40 μm, about 60 μm, about 80 μm, about 100 μm, about 120 μm, about 140 μm, about 160 μm, or about 200 μm. In a specific embodiment, the average diameter of the pores of the substrate and/or coating composition is less than about 10 μm.

In certain embodiments, the average diameter of the molecule of the first material is about ½ to about ⅔ times the average diameter of the pores in the substrate and/or coating composition, and the average diameter of the molecule of the second material is about 1/10 to about ¼ times the average diameter of the pores in the substrate and/or coating composition.

In certain embodiments, the average diameter of a molecule of the first material is greater than the average diameter of a molecule of the second material.

In certain embodiments, the first and/or second material comprises silica, melamine resin, polymethacrylate, polystyrene, polylactide, alumina, or a combination thereof.

In certain embodiments, the first and/or second material is bio-absorbable. In a specific embodiment, the bonded first therapeutic agent is released from the medical device after the first material is absorbed, and the bonded second therapeutic agent is released from the medical device after the second material is absorbed.

In one embodiment, said medical device further comprises a polymeric coating composition comprising a biostable, non-thrombogenic polymeric material. The polymeric coating composition can be disposed on a portion of the substrate or coating composition.

In certain embodiments, the substrate comprises a metal. In specific embodiments, the metal comprises stainless steel, alumina film, platinum, cobalt, chromium, nickel, titanium, magnesium, or a combination thereof.

In certain embodiments, the substrate comprises a polymer. In specific embodiments, the polymer comprises polyethylene, polystyrene, polylactide, or a combination thereof.

In specific embodiments, the first therapeutic agent and/or second therapeutic agent is an anti-proliferative agent comprising rapamycin, daunomycin, mitocycin, dexamethasone, paclitaxel, or a combination thereof; an anti-thrombotic agent comprising heparin, aspirin, warfarin, ticlopidine; an anti-inflammatory agent comprising aspirin, salsalate, diflunisal, ibuprofen, ketoprofen, nabumetone, prioxicam, naproxen, diclofenac, indomethacin, sulindac, tolmetin, etodolac, ketorolac, oxaprozin, celcoxib, glucocorticoids, betamethasone, dexamethasone, prednisolone, corticosterone, budesonide, estrogen, sulfasalazine, rosiglitazone, mycophenolic acid, mesalamine, or a combination thereof; an anti-restenosis agent; or a combination of one or more anti-proliferative agents, anti-thrombotic agents, anti-inflammatory agents, and/or anti-restenosis agents. In preferred embodiments, the first therapeutic agent and/or second therapeutic agent comprises rapamycin, daunomycin, mitocycin, dexamethasone, paclitaxel, everolimus, tacrolimus, zotarolimus, heparin, aspirin, warfarin, ticlopidine, salsalate, diflunisal, ibuprofen, ketoprofen, nabumetone, prioxicam, naproxen, diclofenac, indomethacin, sulindac, tolmetin, etodolac, ketorolac, oxaprozin, celcoxib, or a combination thereof.

The invention also relates to a method for treating or preventing stenosis or restenosis in a subject in need thereof comprising inserting or implanting a medical device of the present invention into the subject, preferably a human subject.

In certain embodiments, the medical device is a stent, such as an intravascular stent.

The invention further relates to methods of preparing the medical device of the present invention. In one embodiment, the method comprises the steps of: (a) providing a medical device comprising a substrate having a surface, (b) creating a plurality of pores in said substrate, (c) bonding a first therapeutic agent to one or more molecule(s) of a first material to form a bonded first therapeutic agent, (d) bonding a second therapeutic agent to one or more molecule(s) of a second material to form a bonded second therapeutic agent, and (e) dispersing said bonded first and second therapeutic agents into the plurality of pores in said substrate. In a related embodiment, the method further comprises the step of: (f) applying a polymeric coating composition comprising a biostable, non-thrombogenic polymeric material on a portion of the surface of the substrate.

In another related embodiment, the method further comprises the steps of: (f) preparing a coating composition comprising said bonded first and second therapeutic agents dispersed therein, and (g) applying said coating composition on a portion of the surface of the substrate. In a related embodiment, the process further comprises the step of: (h) applying a polymeric coating composition comprising a biostable, non-thrombogenic polymeric material on a portion of the coating composition.

In another embodiment, a medical device of the present invention is prepared by a method comprising the steps of: (a) providing a medical device comprising a substrate having a surface, (b) binding a first therapeutic agent to one or more molecule(s) of a first material to form a bonded first therapeutic agent, (c) binding a second therapeutic agent to one or more molecule(s) of a second material to form a bonded second therapeutic agent, (d) preparing a coating composition comprising said bonded first and second therapeutic agents dispersed therein, and (e) applying said coating composition on a portion of the surface of the substrate. In a related embodiment, the method further comprises the step of: (f) applying a polymeric coating composition comprising a biostable, non-thrombogenic polymeric material on a portion of the coating composition.

3.1 Definitions

As used herein, the term “about” is synonymous with the term “approximately,” and refers to a little more or less than the stated value.

As used herein, the term “analogue” refers to a structural derivative of a parent compound that often differs from the parent compound by a single element (e.g., replacement of one atom by another atom or addition/deletion of a functional group).

As used herein, the term “derivative” refers to a compound derived or obtained from a parent compound and containing essential elements of the parent compound.

For more detail of these definitions, see for example, Merriam Webster's Collegiate Dictionary, Tenth Edition, 1997; and The American Heritage® College Dictionary, Third Edition, 2000, which are incorporated herein in their entireties.

### 4. BRIEF DESCRIPTION OF THE DRAWINGS

FIG. 1A-1C show different embodiments of the bonded therapeutic agents of the present invention.

FIG. 2A shows a cross-sectional view of a section of a stent of the present invention comprising a porous substrate comprising a plurality of bonded first and second therapeutic agents.

FIG. 2B shows the stent of FIG. 2A further comprising a polymeric coating composition disposed on the substrate.

FIG. 3A shows a cross-sectional view of a section of a stent of the present invention comprising a substrate and a porous coating composition comprising a plurality of bonded first and second therapeutic agents.

FIG. 3B shows the stent of FIG. 3A further comprising a polymeric coating composition disposed on a portion of the coating composition.

FIG. 4A shows a cross-sectional view of a section of a stent of the present invention comprising a porous substrate comprising a first plurality of bonded first and second therapeutic agents and a porous coating composition comprising a second plurality of bonded first and second therapeutic agents.

FIG. 4B shows the stent of FIG. 4A further comprising a polymeric coating composition disposed on a portion of the coating composition.

### 5. DETAILED DESCRIPTION OF THE INVENTION

The invention is directed to an implantable medical device, preferably a stent, that when in use (e.g., implanted into a body lumen such as a blood vessel), releases different therapeutic agents at different rates. More particularly, the invention is directed to an implantable or insertable medical device comprising a porous substrate and/or coating composition comprising a first therapeutic agent bonded to one or more molecule(s) of a first material to form a bonded first therapeutic agent and a second therapeutic agent bonded to one or more molecule(s) of a second material to form a bonded second therapeutic agent. The first bonded therapeutic agent is released from the medical device (e.g., an intravascular stent) at a rate that is different from the rate that the second bonded therapeutic agent is released from the medical device (e.g., an intravascular stent).

Without being bound by any theory or mechanism, the inventors believe that different therapeutic agents can be released from the claimed medical device at different rates by modifying the sizes, shape, weight, mass, material, and/or nature (e.g., hydrophobic versus hydrophic, anionic versus certiomic, etc) of the therapeutic agents by bonding molecules of different sizes, shape, weight, mass, material, and/or nature to the therapeutic agents. For example, a first therapeutic agent can be bonded to one or more molecule(s) of a first material to form a bonded first therapeutic agent, and a second therapeutic agent can be bonded to one or more molecule(s) of a second material to form a bonded second therapeutic agent. When the medical device is in use (e.g., implanted into a body lumen such as a blood vessel), the larger bonded agent is released from the medical device at a slower rate than the smaller bonded agent. The size of the bonded agent can be measured using methods well known to one skilled in the art. For example, the size of the bonded agent can be measured by average diameter (i.e., the mathematical average of all diameters measured for a non-spherical object), volume, etc. The size of the bonded agent can also be measured by a local property, such as height, optical absorption, or magnetism, using atomic force microscopy (AFM).

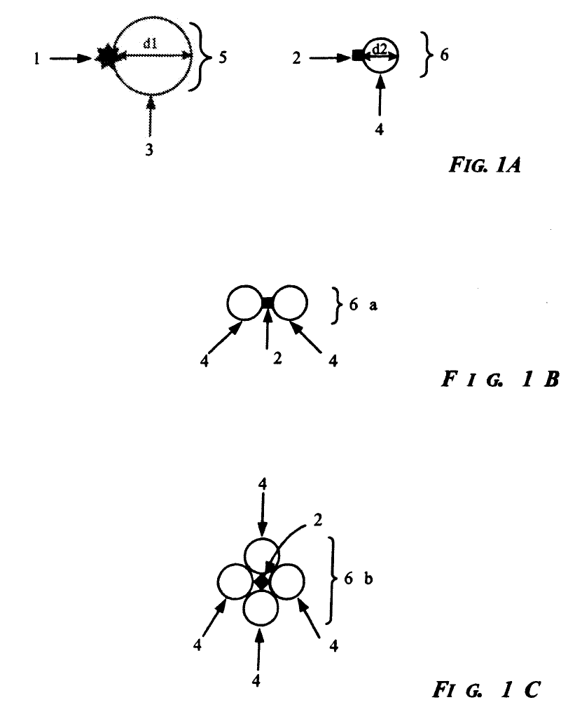

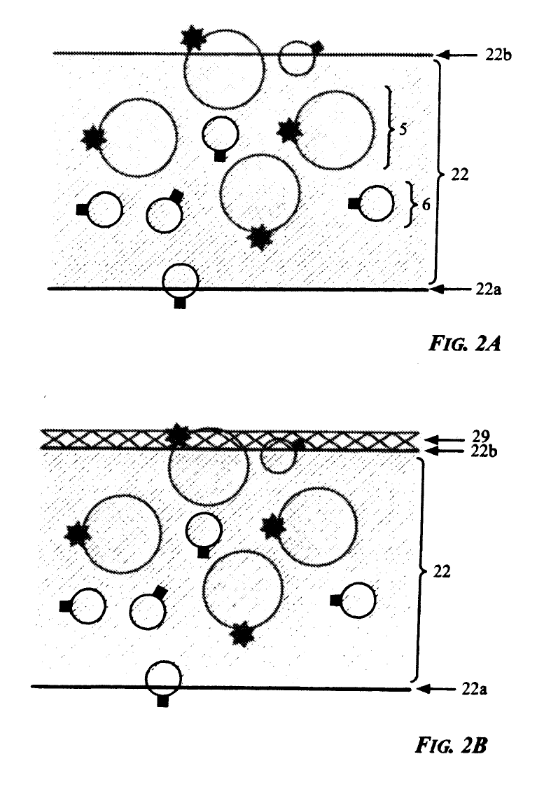

In a specific embodiment, as shown in FIG. 1A, a first therapeutic agent 1 is bonded to a molecule of a first material 3 to form a bonded first therapeutic agent 5, and a second therapeutic agent 2 is bonded to a molecule of a second material 4 to form a bonded second therapeutic agent 6. Assuming that the size of the therapeutic agents is negligible or significantly smaller than the size of the molecule to which they are each bonded, when the average diameter of the molecule of the first material 3 (d1) is greater than the average diameter of the molecule of the second material 4 (d2), the average diameter of the resulting bonded first therapeutic agent 5 is greater than the average diameter of the bonded second therapeutic agent 6, and the resulting bonded first therapeutic agent 5 is greater in size (e.g., volume) than the bonded second therapeutic agent 6.

In another specific embodiment, as shown in FIG. 1B, a second therapeutic agent 2 is bonded to two molecules of the second material 4 to form a bonded second therapeutic agent 6a. Assuming that the size of the therapeutic agents is negligible or significantly smaller than the size of the molecule to which they are each bonded, when the average diameter of the molecule of the first material 3 (d1) is two times the average diameter of the molecule of the second material 4 (d2), the longest diameter (i.e., the longest diameter measured for a non-spherical object) of the resulting bonded first therapeutic agent 5 is equal to the average diameter of the bonded second therapeutic agent 6, but the resulting bonded first therapeutic agent 5 is smaller in size (e.g., volume) than the bonded second therapeutic agent 6.

In yet another specific embodiment, as shown in FIG. 1C, a second therapeutic agent 2 is bonded to four molecules of the second material 4 to form a bonded second therapeutic agent 6b. Assuming that the size of the therapeutic agents is negligible or significantly smaller than the size of the molecule to which they are each bonded, when the average diameter of the molecule of the first material 3 (d1) is two times the average diameter of the molecule of the second material 4 (d2), the longest diameter of the resulting bonded first therapeutic agent 5 is equal to the average diameter of the bonded second therapeutic agent 6, but the resulting bonded first therapeutic agent 5 is greater in size (e.g., volume) than the bonded second therapeutic agent 6.

In one embodiment, the medical device of the present invention comprises a substrate comprising a plurality of bonded first therapeutic agents and a plurality of bonded second therapeutic agents, wherein when the medical device is in use (e.g., implanted into a body lumen such as a blood vessel), the bonded first therapeutic agents are released at a first rate and the bonded second therapeutic agents are released at a second rate that is faster than said first rate. In specific embodiments, the second rate can be about ten times, nine times, eight times, seven times, six times, five times, four times, three times, or two times faster than the first rate.

FIG. 2A shows a cross-sectional view of a stent strut comprising a porous substrate 22 comprising a first therapeutic agent 24 bonded to a molecule of a first material 23 to form a bonded first therapeutic agent 25, and a second therapeutic agent 27 bonded to a molecule of a second material 26 to form a bonded second therapeutic agent 28. As shown in FIG. 2A, the bonded first or second therapeutic agent can extend beyond the surface of the substrate 22a and 22b.

In certain embodiments, the medical device further comprises a polymeric coating composition disposed on a portion of the substrate. Preferably, the polymeric coating composition comprises a biostable, non-thrombogenic polymeric material, and wherein the bonded first therapeutic agent is released from the medical device at a third rate that is different from the first rate, and the bonded second therapeutic agent is released from the medical device at a fourth rate that is different from the second rate.

For example, a polymeric coating composition 29 comprising a biostable, non-thrombogenic polymer material can be disposed on a portion of the substrate 22, as shown in FIG. 2B. This polymeric coating composition 29 can modify the release of both the bonded first and second therapeutic agents, such as by decreasing the amount and/or rate of release of both such bonded agents (e.g., reduces so-called “burst effects”).

In another embodiment, the medical device of the present invention comprises a substrate and a coating composition disposed on at least a portion of the substrate, wherein the coating composition comprises a plurality of bonded first therapeutic agents and bonded second therapeutic agents, wherein when the medical device is in use (e.g., implanted into a body lumen such as a blood vessel), the bonded first therapeutic agents are released at a first rate and the bonded second therapeutic agents are released at a second rate that is faster than said first rate.

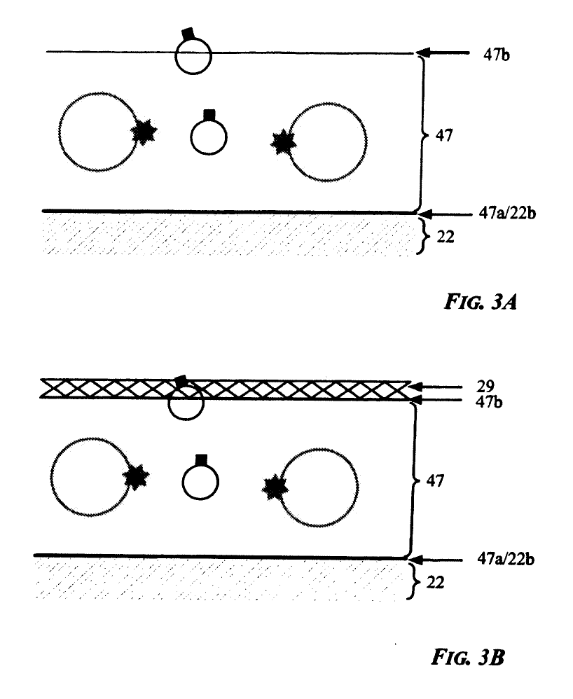

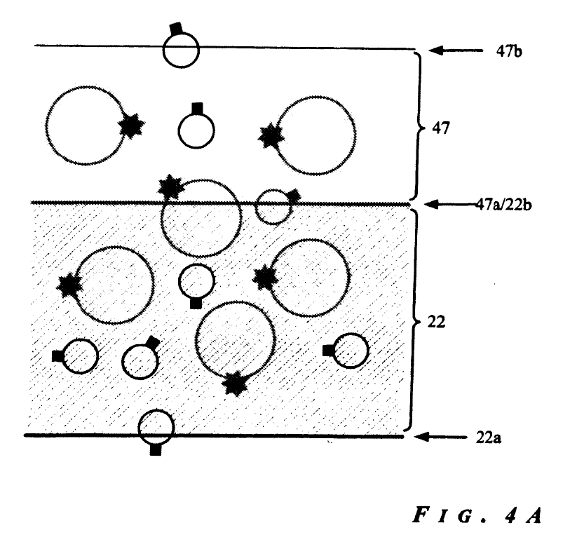

FIG. 3A shows a stent comprising a substrate 49 coated with a porous coating composition 47 comprising a plurality of bonded first therapeutic agents 25 having an average diameter D₁ and bonded second therapeutic agents 28 having an average diameter D₂. As shown in FIG. 3A, the bonded first or second therapeutic agent can extend beyond the surface of the coating composition. The polymeric coating 29 can be disposed on a portion of the coating composition 47, as shown in FIG. 3B to modify the release of the bonded therapeutic agents.

In yet another embodiment, the medical device of the present invention comprises a substrate and a coating composition disposed on at least a portion of the substrate, wherein the substrate comprise a first plurality of bonded first therapeutic agents and bonded second therapeutic agents, and the coating composition comprises a second plurality of bonded first therapeutic agents and bonded second therapeutic agents, wherein when the medical device is in use (e.g., implanted into a body lumen such as a blood vessel), the bonded first therapeutic agents are released at a first rate and the bonded second therapeutic agents are released at a second rate that is faster than said first rate.

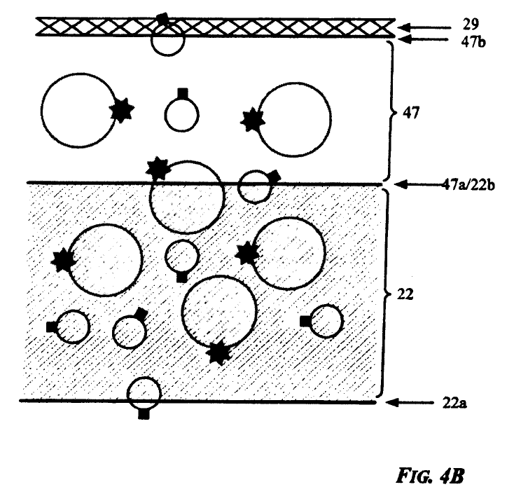

FIG. 4A shows a stent comprising a porous substrate 22 and a porous coating composition 47 disposed on a portion of the porous substrate 22, wherein the substrate 22 and coating composition 47 both comprise bonded first therapeutic agents 25 having an average diameter of D₁ and bonded second therapeutic agents 28 having an average diameter of D₂. As shown in FIG. 4A, the bonded first or second therapeutic agent can extend beyond the surface of the substrate and/or coating composition. The polymeric coating 29 can be disposed on a portion of the coating composition 47, as shown in FIG. 4B, to modify the release of the bonded therapeutic agents.

The medical devices of the present invention are discussed in more detail in Section 5.1 infra. Methods of preparing and using the medical device of the present invention are discussed in Sections 5.2 and 5.3, respectively, infra. For clarity of disclosure, and not by way of limitation, the detailed description of the invention is divided into the subsections which follow.

5.1 The Medical Device

5.1.1. Bonded Therapeutic Agents

The medical devices of the present invention comprise bonded therapeutic agents, dispersed into the pores of a porous substrate and/or a porous coating composition. The bonded therapeutic agents are released from the pores of the medical device when the medical device is implanted or inserted into the body of a patient. The average diameter of the bonded therapeutic agents, coupled with the average diameter of the pores of the substrate and/or coating composition determine the rate at which the bonded therapeutic agents are released from the medical device.

5.1.1.1 Therapeutic Agents

In certain embodiments, the therapeutic agent is useful for inhibiting cell proliferation, contraction, migration, hyperactivity, or addressing other conditions. As used herein, the term “therapeutic agent” encompasses drugs, genetic materials and biological materials. Non-limiting examples of suitable therapeutic agent include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, taxol, paclitaxel, 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, 2′-O-ester with N-dimethylaminoethyl)glutamide hydrochloride salt, nitroglycerin, nitrous oxides, nitric oxides, antibiotics, aspirins, digitalis, estrogen, estradiol and glycosides. In a preferred embodiment, the therapeutic agent is taxol (e.g., Taxol®), or its analogues or derivatives. In another preferred embodiment, the therapeutic agent is paclitaxel. In yet another preferred embodiment, the therapeutic agent is an antibiotic including, but not limited to, erythromycin, amphotericin, rapamycin, adriamycin.

The term “genetic materials” means DNA or RNA, including, without limitation, of DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include, but are not limited to, vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (IL) (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include, but are not limited to, progenitor cells (e.g., endothelial progenitor cells), stem cells (e.g., mesenchymal, hematopoietic, neuronal), stromal cells, parenchymal cells, undifferentiated cells, fibroblasts, macrophage, satellite cells.

Other therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid, and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, taxol and its analogues or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
vascular cell growth promotors such as growth factors, Vascular Endothelial Growth Factors (FEGF, all types including VEGF-2), growth factor receptors, transcriptional activators, and translational promotors;
DNA demethylating drugs such as 5-azacytidine, which is also categorized as a RNA or DNA metabolite that inhibit cell growth and induce apoptosis in certain cancer cells;
vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents; vasodilating agents; and agents which interfere with endogenous vasoactive mechanisms;
anti-oxidants, such as probucol;
antibiotic agents, such as penicillin, cefoxitin, oxacillin, tobramycin;
macrolides such as sirolimus (rapamycin), everolimus, tacrolimus, pimecrolimus, and zotarolimus;
angiogenic substances, such as acidic and basic fibroblast growth factors, estrogen including estradiol (E2), estriol (E3) and 17-beta estradiol; and
drugs for heart failure, such as digoxin, beta-blockers, angiotensin-converting enzyme (ACE) inhibitors including captopril and enalopril, statins and related compounds.

Preferred therapeutic agents include anti-proliferative drugs such as steroids, vitamins, and restenosis-inhibiting agents. Preferred anti-restenosis agents include microtubule stabilizing agents such as Taxol®, paclitaxel (i.e., paclitaxel, paclitaxel analogues, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, and 2′-O-ester with N-(dimethylaminoethyl)glutamide hydrochloride salt.

Other preferred therapeutic agents include nitroglycerin, nitrous oxides, nitric oxides, antibiotics, aspirins, digitalis, estrogen derivatives such as estradiol and glycosides.

In preferred embodiments, the therapeutic agent comprises rapamycin, daunomycin, mitocycin, dexamethasone, paclitaxel, everolimus, tacrolimus, zotarolimus, heparin, aspirin, warfarin, ticlopidine, salsalate, diflunisal, ibuprofen, ketoprofen, nabumetone, prioxicam, naproxen, diclofenac, indomethacin, sulindac, tolmetin, etodolac, ketorolac, oxaprozin, celcoxib, or a combination thereof.

The therapeutic agents can be synthesized by methods well known to one skilled in the art. Alternatively, the therapeutic agents can be purchased from chemical and pharmaceutical companies.

5.1.1.2 First and Second Materials

The rate a therapeutic agent is released from a medical device of the present invention can be adjusted based on the size (e.g., average diameter, volume), mass and/or nature of the molecule to which it is being bonded.

In certain embodiments, the molecule bonded to the therapeutic agent can be of a material comprising silica, melamine resin, polymethacrylate, polystyrene, polylactide, alumina, or a combination thereof.

In certain embodiments, the material is at least partially (e.g., at least 30%, 40%, 50%, 60%, 70%, 80%, 90%, 95%, 97%, or 99%) or completely (i.e., 100%) bio-absorbable such that, upon exposure to a body tissue, the material decomposes, leaving behind the therapeutic agent to interact with the tissue such as the vessel wall. In a specific embodiment, the therapeutic agent is released from the medical device after the material is absorbed.

In certain embodiments, the average diameter of a molecule of a first material is greater than the average diameter of a molecule of a second material.

In certain embodiments, the average diameter of a molecule of a first material is about ½ to about ⅔ times the average diameter of the pores in the substrate and/or coating composition, and the average diameter of a molecule of a second material is about 1/10 to about ¼ times the average diameter of the pores in the substrate and/or coating composition.

In specific embodiments, the average diameter of a molecule of a material ranges from 0.001 μm to 130 μm. In specific embodiments, the average diameter of a molecule of a first material ranges from about 0.001 μm to about 100 μm, about 0.01 μm to about 80 μm, about 0.1 μm to about 60 μm, about 1 μm to about 40 μm, about 10 μm to about 30 μm, and the average diameter of a molecule of a second material ranges from about 0.001 μm to about 40 μm, about 0.01 μm to about 30 μm, about 0.1 μm to about 20 μm, about 1 μm to about 10 μm. In certain embodiments, the average diameter of a molecule of a first material is about 0.001 μm, about 0.01 μm, about 0.1 μm, about 1 μm, about 10 μm, about 20 μm, about 40 μm, about 60 μm, about 80 μm, about 100 μm, or about 130 μm, and the average diameter of a molecule of a second material is about 0.001 μm, about 0.01 μm, about 0.1 μm, about 1 μm, about 10 μm, about 20 μm, about 30 μm, about 40 μm, or about 50 μm.

The diameter of the materials can be measured by any methods known to one skilled in the art, including, but not limited to, microparticle measurement techniques comprising transmission electron microscopy, scanning electron microscopy, optical microscopy, and particle size analyzer, as outlined by standard microparticle measurement techniques from the National Institute of Standards and Technology.

The materials can be synthesized by methods well known to one skilled in the art. Alternatively, the materials can be purchased from chemical and pharmaceutical companies.

5.1.2. Types of Medical Device

The medical devices of the present invention can be implanted or inserted into the body of a patient. Medical devices suitable for the present invention include, but are not limited to, stents, surgical staples, catheters, such as balloon catheters, central venous catheters, and arterial catheters, guidewires, cannulas, cardiac pacemaker leads or lead tips, cardiac defibrillator leads or lead tips, implantable vascular access ports, blood storage bags, blood tubing, vascular or other grafts, intra aortic balloon pumps, heart valves, cardiovascular sutures, total artificial hearts and ventricular assist pumps, and extra corporeal devices such as blood oxygenators, blood filters, septal defect devices, hemodialysis units, hemoperfusion units and plasmapheresis units.

Medical devices suitable for the present invention include, but are not limited to, those that have a tubular or cylindrical like portion. For example, the tubular portion of the medical device need not be completely cylindrical. The cross section of the tubular portion can be any shape, such as rectangle, a triangle, etc., not just a circle. Such devices include, but are not limited to, stents, balloon catheters, and grafts. A bifurcated stent is also included among the medical devices which can be fabricated by the method of the present invention. In one embodiment the medical device is a stent having a sidewall comprising a plurality of struts defining a plurality of openings. In some embodiments, the stent has an open lattice sidewall stent structure made up of openings and struts. The medical device has an outer surface that is adapted for exposure to a body lumen, an inner surface, and at least one side surface between the outer surface and the inner surface.

In addition, the tubular portion of the medical device may be a sidewall that may comprise a plurality of struts defining a plurality of openings. The sidewall defines a lumen. The struts may be arranged in any suitable configuration. Also, the struts do not all have to have the same shape or geometric configuration. When the medical device is a stent comprising a plurality of struts, the surface is located on the struts. Each individual strut has an outer surface adapted for exposure to the body tissue of the patient, an inner surface, and at least one side surface between the outer surface and the inner surface.

Medical devices that are particularly suitable for the present invention include any kind of stent for medical purposes which is known to the skilled artisan. Preferably, the stents are intravascular stents that are designed for permanent implantation in a blood vessel of a patient. In certain embodiments, the stent comprises an open lattice sidewall stent structure. In preferred embodiments, the stent suitable for the present invention is an Express stent. More preferably, the Express stent is an Express™ stent or an Express2™ stent (Boston Scientific, Inc. Natick, Mass.). Other suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al.

The framework of suitable stents may be formed through various methods as known in the art. The framework may be welded, molded, laser cut, electro-formed, or consist of filaments or fibers which are wound or braided together in order to form a continuous structure.

Suitable substrate of the medical device (e.g., stents) of the present invention may be fabricated from a metallic material, ceramic material, polymeric or non-polymerical material, or a combination thereof (see Sections 5.1.2.1 to 5.1.2.4 infra.). Preferably, the materials are biocompatible. The material may be porous or non-porous, and the porous structural elements can be microporous or nanoporous.

5.1.2.1 Metallic Materials for Device Formation

In certain embodiments, the medical device of the present invention comprises a substrate which is metallic. Suitable metallic materials useful for making the substrate include, but are not limited to, metals and alloys based on titanium (such as nitinol, nickel titanium alloys, thermo memory alloy materials), stainless steel, gold, platinum, iridium, molybdenum, niobium, palladium, chromium, tantalum, nickel chrome, or certain cobalt alloys including cobalt chromium nickel alloys such as Elgiloy® and Phynox®, or a combination thereof. Other metallic materials include clad composite filaments, such as those disclosed in WO 94/16646.

In certain embodiments, the substrate comprises a metal oxide. Suitable metal oxides include, but are not limited to, transition metal oxides, platinum oxide, tantalum oxide, titanium oxide, titanium dioxide, iridium oxide, niobium oxide, zirconium oxide, tungsten oxide, rhodium oxide, or a combination thereof Preferably, the metal or metal oxide is biocompatible.

Preferably, the metal or metal oxide region comprises a radiopaque material. Including a radiopaque material may be desired so that the medical device is visible under X-ray or fluoroscopy. Suitable materials that are radiopaque include, but are not limited to, gold, tantalum, platinum, bismuth, iridium, zirconium, iodine, titanium, barium silver, tin, alloys of these metals, or a combination thereof.

Furthermore, although the invention can be practiced by using a single type of metal to form the substrate, various combinations of metals can also be employed. The appropriate mixture of metals can be coordinated to produce desired effects when incorporated into a substrate.

5.1.2.2 Ceramic Materials for Device Formation

In certain embodiments, the medical device of the present invention comprises a substrate which is ceramic. Suitable ceramic materials used for making the substrate include, but are not limited to, oxides, carbides, or nitrides of the transition elements such as titanium oxides, hafnium oxides, iridium oxides, chromium oxides, aluminum oxides, zirconium oxides, or a combination thereof. Silicon based materials, such as silica, may also be used.

Furthermore, although the invention can be practiced by using a single type of ceramic to form the substrate, various combinations of ceramics can also be employed. The appropriate mixture of ceramics can be coordinated to produce desired effects when incorporated into a substrate.

5.1.2.3 Polymeric Materials for Device Formation

In certain embodiments, the medical device of the present invention comprises a substrate which is polymeric. The polymer(s) useful for forming the components of the medical devices should be ones that are biocompatible and avoid irritation to body tissue. The polymers can be biostable or bioabsorbable. Suitable polymeric materials useful for making the substrate include, but are not limited to, isobutylene-based polymers, polystyrene-based polymers, polyacrylates, and polyacrylate derivatives, vinyl acetate-based polymers and its copolymers, polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, chitins, or a combination thereof.

Other polymers that are useful as materials for making the substrate include, but are not limited to, dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(ε-caprolactone), poly(β-hydroxybutyrate), polydioxanone, poly(γ-ethyl glutamate), polyiminocarbonates, poly(ortho ester), polyanhydrides, styrene isobutylene styrene, polyetheroxides, polyvinyl alcohol, polyglycolic acid, polylactic acid, polyamides, poly-2-hydroxybutyrate, polycaprolactone, poly(lactic-co-clycolic)acid, Teflon, alginate, dextran, chitin, cotton, polyglycolic acid, polyurethane, derivatized versions thereof, (i.e., polymers which have been modified to include, for example, attachment sites or cross-linking groups, e.g., arginine-glycine-aspartic acid RGD, in which the polymers retain their structural integrity while allowing for attachment of cells and molecules, such as proteins and/or nucleic acids), or a combination thereof.

The polymers may be dried to increase their mechanical strength. The polymers may then be used as the base material to form a whole or part of the substrate.

Furthermore, although the invention can be practiced by using a single type of polymer to form the substrate, various combinations of polymers can also be employed. The appropriate mixture of polymers can be coordinated to produce desired effects when incorporated into a substrate.

5.1.2.4 Non-Polymeric Materials for Device Formulation

In certain embodiments, the medical device of the present invention comprises a substrate which is non-polymeric. Suitable non-polymeric materials useful for making the substrate include, but are not limited to, sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine(lecithin), phosphatidylserine, phosphatidylethanolamine, phosphatidylinositol, and lysoderivatives thereof; sphingosine and derivatives thereof; sphingomyelins such as stearyl, palmitoyl, and tricosanyl sphingomyelins; ceramides such as stearyl and palmitoyl ceramides; glycosphingolipids; lanolin and lanolin alcohols; or a combination thereof. Preferred non-polymers include cholesterol, glyceryl monostearate, glycerol tristearate, stearic acid, stearic anhydride, glyceryl monooleate, glyceryl monolinoleate, and acetylated monoglycerides.

Furthermore, although the invention can be practiced by using a single type of non-polymer to form the substrate, various combinations of non-polymers can also be employed. The appropriate mixture of non-polymers can be coordinated to produce desired effects when incorporated into a substrate.

5.1.2.5 Porous Substrate

In certain embodiments, the substrate of the medical device of the present invention is porous and the bonded therapeutic agents discussed in Section 5.1.1 supra. can be dispersed into the pores of the porous substrate. In specific embodiments, the composition forming the substrate comprises at least 1%, at least 5%, at least 10%, at least 20%, at least 30%, at least 40%, at least 50%, at least 60%, at least 70%, at least 80%, at least 90%, at least 95%, at least 97%, at least 99% or more by weight of bonded therapeutic agents. The pores in the substrate can be connected to or in communication with the outer surface of the substrate. Also, the pores may be discrete, interconnected, or disposed in a pattern. In addition, the pores may have any shape or size, including pores shaped like channels, void pathways or microscopic conduits. The average diameter of the pores in the substrate is within a range of about 0.01 μm to about 200 μm, about 0.1 μm to about 180 μm, about 0.5 μm to about 160 μm, about 1 μm to about 140 μm, about 10 μm to about 120 μm, about 20 μm to about 100 μm, about 30 μm to about 80 μm, about 40 μm to about 60 μm. In certain embodiments, the average diameter of the pores in the substrate is about 0.01 μm, about 0.1 μm, about 1 μm, about 10 μm, about 20 μm, about 40 μm, about 60 μm, about 80 μm, about 100 μm, about 120 μm, about 140 μm, about 160 μm, about 200 μm. In one embodiment, the average diameter of the pores of the substrate is less than about 10 μm.

5.1.3. Coating Compositions

In certain embodiments, the medical device of the present invention comprises a coating composition and/or a polymeric coating composition. One or more polymers may be used to form the coating composition or the polymeric coating composition. The polymers should be ones that are biocompatible and avoid irritation to body tissue. The polymers can be either biostable or bioabsorbable. Suitable polymers include those discussed in Section 5.1.2.3 supra. that are used to fabricate the medical devices of the present invention.

Other suitable polymers useful for making the coating composition and porous coating composition include, but are not limited to, isobutylene styrene copolymers, thermoplastic elastomers in general, polyolefins, polyisobutylene, ethylene alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene methyl methacrylate copolymers, acrylonitrile styrene copolymers, ABS (acrylonitrile butadiene styrene) resins, ethylene vinyl acetate copolymers, polyamides such as Nylon 66 and polycaprolactone, alkyd resins, polycarbonates, polyoxymethylenes, polyimides, polyethers, epoxy resins, rayon triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid polyethylene oxide copolymers, EPDM (ethylene propylene diene) rubbers, fluorosilicones, polyethylene glycol, polysaccharides, phospholipids, or a combination thereof.

Preferably, for medical devices which undergo mechanical challenges, (e.g., expansion and contraction), polymers should be selected from elastomeric polymers such as silicones (e.g., polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating compositions and polymeric coating compositions are capable of undergoing deformation under the yield point when the device is subjected to forces, stress or mechanical challenge.

Examples of preferred adhesive polymers include, but are not limited to, copolymers of styrene and isobutylene, cyanoacrylate or ethylene vinyl acetate, isobutylene-based polymers, acrylate-based polymers, fibrin, or combinations thereof.

Hydrogel polymers such as polyhema, polyethylene glycol, polyacrylamide, and other acrylic hydrogels may also be used. Other hydrogel polymers that may be used are disclosed in U.S. Pat. No. 5,304,121 to Sahatjian, U.S. Pat. No. 5,464,650 to Berg et al., U.S. Pat. No. 6,368,356 to Zhong et al., PCT publication WO 95/03083 to Sahatjian et al., and U.S. Pat. No. 5,120,322 to Davis et al., which are incorporated by references herein in their entireties.

Solvents used to prepare the coating composition and polymeric coating composition include ones which can dissolve or suspend the polymeric material in solution. Examples of suitable solvents include, but are not limited to, tetrahydrofuran, methylethylketone, chloroform, toluene, acetone, isooctane, 1,1,1-trichloroethane, dichloromethane, isopropanol, IPA, or a combination thereof.

5.1.3.1 Porous Coating Composition

In certain embodiments, the coating composition is porous and the bonded therapeutic agents discussed in Section 5.1.1 supra. can be dispersed into the pores of the porous coating composition. In specific embodiments, the composition forming the coating composition comprises at least 1%, at least 5%, at least 10%, at least 20%, at least 30%, at least 40%, at least 50%, at least 60%, at least 70%, at least 80%, at least 90%, at least 95%, at least 97%, at least 99% or more by weight of bonded therapeutic agents. The pores in the coating composition can be connected to or in communication with the outer surface of the coating composition. Also, the pores may be discrete, interconnected, or disposed in a pattern. In addition, the pores may have any shape or size, including pores shaped like channels, void pathways or microscopic conduits. The average diameter of the pores in the coating composition is within a range of about 0.01 μm to about 200 μm, about 0.1 μm to about 180 μm, about 0.5 μm to about 160 μm, about 1 μm to about 140 μm, about 10 μm to about 120 μm, about 20 μm to about 100 μm, about 30 μm to about 80 μm, about 40 μm to about 60 μm. In certain embodiments, the average diameter of the pores in the coating composition is about 0.01 μm, about 0.1 μm, about 1 μm, about 10 μm, about 20 μm, about 40 μm, about 60 μm, about 80 μm, about 100 μm, about 120 μm, about 140 μm, about 160 μm, about 200 μm. In one embodiment, the average diameter of the pores of the coating composition is less than about 10 μm.

5.1.3.2 Polymeric Costing Composition

In certain embodiments, the medical device of the present invention further comprises a polymeric coating composition disposed on a portion of a substrate or a coating composition. In a preferred embodiment, the polymeric coating composition comprises a biostable, non-thrombogenic polymeric material. The presence of the polymeric coating composition can change the release rate of the bonded therapeutic agents (e.g., reduces so-called “burst effects”) such that bonded therapeutic agent is released from the medical device at a rate that is different from the rate it is released from a medical device without the polymer coating composition.

In certain embodiments, the polymers used to form the polymeric coating composition can be the same or different as the polymers used to form the coating composition.

5.2 Method of Making the Medical Device

The invention also relates to methods of making the medical devices of the invention. In certain embodiments, the method comprises the steps of: (a) providing a medical device comprising a substrate having a surface, (b) creating a plurality of pores in said substrate, (c) binding a first therapeutic agent to one or more molecule(s) of a first material to form a bonded first therapeutic agent, (d) binding a second therapeutic agent to one or more molecule(s) of a second material to form a bonded second therapeutic agent, and (e) dispersing said bonded first and second therapeutic agents into the plurality of pores in said substrate. In another embodiment, the method further comprises the step of: (f) applying a polymeric coating composition comprising a biostable, non-thrombogenic polymeric material on a portion of the surface of the substrate. In another related embodiment, the method further comprises the steps of: (f) preparing a coating composition comprising said bonded first and second therapeutic agents dispersed therein, and (g) applying said coating composition on a portion of the surface of the substrate. The method can further comprise the step of: (h) applying a polymeric coating composition comprising a biostable, non-thrombogenic polymeric material on a portion of the coating composition.

In certain other embodiments, the method comprises the steps of: (a) binding a first therapeutic agent to one or more molecule(s) of a first material to form a bonded first therapeutic agent; (b) binding a second therapeutic agent to one or more molecule(s) of a second material to form a bonded second therapeutic agent; and (c) mixing the bonded first and second therapeutic agents with a composition to form a substrate. In a related embodiment, the method further comprises the step of; and (d) applying a polymeric coating composition comprising a biostable, non-thrombogenic polymeric material on a portion of the surface of the substrate. In another related embodiment, the method further comprises the steps of: (d) preparing a coating composition comprising said bonded first and second therapeutic agents dispersed therein, and (e) applying said coating composition on a portion of the surface of the substrate. The method can further comprise the step of: (f) applying a polymeric coating composition comprising a biostable, non-thrombogenic polymeric material on a portion of the coating composition.

In certain other embodiments, the medical device is prepared by a method comprising the steps of: (a) providing a medical device comprising a substrate having a surface, (b) binding a first therapeutic agent to one or more molecule(s) of a first material to form a bonded first therapeutic agent, (c) binding a second therapeutic agent to one or more molecule(s) of a second material to form a bonded second therapeutic agent, (d) preparing a coating comprising said bonded first and second therapeutic agents dispersed therein, and (e) applying said coating on a portion of the surface of the substrate. In a related embodiment, the method further comprises the step of: (f) applying a polymeric coating composition comprising a biostable, non-thrombogenic polymeric material on a portion of the coating composition.

5.2.1. Method of Preparing a Bonded Therapeutic Agent

The therapeutic agents discussed in Section 5.1.1.1 supra. can be bonded to one or more molecule(s) of a material discussed in Section 5.1.1.2 supra. by any method known to one skilled in the art including, but not limited to, ionic bonds, hydrogen bonds, covalent or non-covalent chemical associations, (i.e., hydrophobic as through van der Waals forces or charge-charge interactions), or a combination thereof. The strength of the bond can be measured by any method known to one skilled in the art

In specific embodiments, the average diameter of a molecule of a material ranges from 0.001 μm to 130 μm. In specific embodiments, the average diameter of a molecule of a first material ranges from about 0.001 μm to about 100 μm, about 0.01 μm to about 80 μm, about 0.1 μm to about 60 μm, about 1 μm to about 40 μm, about 10 μm to about 30 μm, and the average diameter of a molecule of a second material ranges from about 0.001 μm to about 40 μm, about 0.01 μm to about 30 μm, about 0.1 μm to about 20 μm, about 1 μm to about 10 μm. In certain embodiments, the average diameter of a molecule of a first material is about 0.001 μm, about 0.01 μm, about 0.1 μm, about 1 μm, about 10 μm, about 20 μm, about 40 μm, about 60 μm, about 80 μm, about 100 μm, or about 130 μm, and the average diameter of a molecule of a second material is about 0.001 μm, about 0.01 μm, about 0.1 μm, about 1 μm, about 10 μm, about 20 μm, about 30 μm, about 40 μm, or about 50 μm.

5.2.2. Method of Preparing a Porous Substrate Comprising a Bonded Therapeutic Agent

A substrate of the present invention can be porous and comprises the bonded therapeutic agents discussed in Section 5.1.1 supra. The size and number of pores in the substrate can effect the release rate of the bonded therapeutic agents. For example, a substrate with larger pores will allow the bonded therapeutic agent to be released more quickly than a substrate with smaller pores. A more porous substrate will allow a greater number of the bonded therapeutic agents to be released than a less porous substrate.

The pores of the substrate can be created by any method known to one skilled in the art including, but not limited to, sintering, codeposition, micro-roughing, or a combination thereof For example, the porous structure can be made by a deposition process such as sputtering and adjusting the deposition condition, by micro roughing using reactive plasmas, by ion bombardment electrolyte etching, or a combination thereof. Other methods include, but are not limited to, alloy plating, physical vapor deposition, chemical vapor deposition, sintering, or a combination thereof.

The bonded therapeutic agents can be dispersed in the pores of the substrate by any method known to one skilled in the art including, but not limited to, dip coating, spray coating, spin coating, plasma deposition, condensation, electrochemically, electrostatically, evaporation, plasma vapor deposition, cathodic arc deposition, sputtering, ion implantation, use of a fluidized bed, or a combination thereof. Methods suitable for dispersing the bonded therapeutic agents to the substrate of the present invention preferably do not alter or adversely impact the therapeutic properties of the therapeutic agent.

5.2.3. Method of Preparing a Porous Coating Composition Comprising a Bonded Therapeutic Agent

A coating composition of the present invention can be porous and comprises the bonded therapeutic agents discussed in Section 5.1.1 supra. The size of pores in the coating composition can effect the release rate of the bonded therapeutic agents. For example, a coating composition with larger pores will allow bonded therapeutic agent to be released more quickly than a coating composition with smaller pores. A more porous coating composition will allow a greater number of bonded therapeutic agents to be released than a less porous coating composition.

The pores of the coating composition can be created by any method known to one skilled in the art including, but not limited to, vacuum plasma spraying on the coating comprising a first metal with process parameters that promote the formation of porosity. The pore size could be varied by the amount of gas entrapped in the coating.

The bonded therapeutic agents can be dispersed in the pores of the substrate by any suitable method including, but not limited to, dip coating, spray coating, spin coating, plasma deposition, condensation, electrochemically, electrostatically, evaporation, plasma vapor deposition, cathodic arc deposition, sputtering, ion implantation, use of a fluidized bed, or a combination thereof. Methods suitable for dispersing the bonded therapeutic agents to the coating composition of the present invention preferably do not alter or adversely impact the therapeutic properties of the therapeutic agent.

5.2.4. Method of Applying the Coating Composition

The coating composition can be applied to at least a portion of a surface of a medical device by any method known to one skilled in the art, including, but not limited to, dipping, spraying, such as by conventional nozzle or ultrasonic nozzle, laminating, pressing, brushing, swabbing, dipping, rolling, electrostatic deposition, painting, electroplating, evaporation, plasma-vapor deposition, a batch process such as air suspension, pan coating or ultrasonic mist spraying, cathodic-arc deposition, sputtering, ion implantation, electrostatically, electroplating, electrochemically, and all modern chemical ways of immobilization of bio-molecules to surfaces, or a combination thereof. Preferably, the coating composition is applied by spraying, rolling, laminating, pressing, or a combination thereof.

More than one coating method can be used to apply the coating composition. If more than one coating composition is applied, the coating compositions can be applied by the same or different methods. Such methods are commonly known to the skilled artisan.

### 6. EQUIVALENTS

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

What is claimed is:

1. An intravascular stent comprising:
a substrate comprising a first therapeutic agent and a second therapeutic agent;
wherein the first therapeutic agent is bonded to one or more molecule(s) of a first material to form a bonded first therapeutic agent, and the second therapeutic agent is bonded to one or more molecule(s) of a second material to form a bonded second therapeutic agent,
wherein said bonded first therapeutic agent is greater in size than said bonded second therapeutic agent, and
wherein when the stent is implanted into a blood vessel, the bonded first therapeutic agent is released from the stent at a first rate and the bonded second therapeutic agent is released from the stent at a second rate that is faster than said first rate.

2. The stent of claim 1, wherein said substrate comprises stainless steel, alumina film, platinum, cobalt, chromium, nickel, titanium, magnesium, or a combination thereof.

3. The stent of claim 1, wherein said substrate comprises polyethylene, polystyrene, polylactide, or a combination thereof.

4. The stent of claim 1, wherein said substrate is porous.

5. The stent of claim 1, wherein at least one of the first or second material comprises silica, melamine resin, polymethacrylate, polystyrene, polylactide, alumina, or a combination thereof.

6. The stent of claim 1, wherein said first material and said second material are bioabsorbable, and wherein said bonded first therapeutic agent is released from the stent after the first material is at least partially absorbed, and wherein said bonded second therapeutic agent is released from the stent after the second material is at least partially absorbed.

7. The stent of claim 1, wherein the average diameter of the molecule(s) of the first material is greater than the average diameter of the molecule(s) of the second material,

8. The stent of claim 1, wherein at least one of the first or second therapeutic agent comprises an anti-proliferative agent, anti-thrombogenic agent, anti-inflammatory agent, or a combination thereof.

9. The stent of claim 1, wherein at least one of the first or second therapeutic agent comprises rapamycin, daunomycin, mitocycin, dexamethasone, paclitaxel, everolimus, tacrolimus, zotarolimus, heparin, aspirin, warfarin, ticlopidine, salsalate, diflunisal, ibuprofen, ketoprofen, nabumetone, prioxicam, naproxen, diclofenac, indomethacin, sulindac, tolmetin, etodolac, ketorolac, oxaprozin, celcoxib, or a combination thereof.

10. The stent of claim 1, further comprising:
a polymeric coating composition disposed on said substrate,
wherein said polymeric coating composition comprises a biostable, non-thrombogenic polymeric material, and wherein when the stent is implanted into a blood vessel, the bonded first therapeutic agent is released from the stent at a third rate that is different from the first rate, and the bonded second therapeutic agent is released from the stent at a fourth rate that is different from the second rate.

11. An intravascular stent comprising:
a substrate; and
a coating composition disposed on at least a portion of the substrate, wherein the coating composition comprises a first therapeutic agent and a second therapeutic agent;
wherein the first therapeutic agent is bonded to one or more molecule(s) of a first material to form a bonded first therapeutic agent, and the second therapeutic agent is bonded to one or more molecule(s) of a second material to form a bonded second therapeutic agent,
wherein said bonded first therapeutic agent is greater in size than said bonded second therapeutic agent, and
wherein when the stent is implanted into a blood vessel, the bonded first therapeutic agent is released from the stent at a first rate and the bonded second therapeutic agent is released from the stent at a second rate that is faster than said first rate.

12. The stent of claim 11, wherein said substrate comprises stainless steel, alumina film, platinum, cobalt, chromium, nickel, titanium, magnesium, or a combination thereof.

13. The stent of claim 11, wherein said substrate comprises polyethylene, polystyrene, polylactide, or a combination thereof.

14. The stent of claim 11, wherein said at least one of the substrate or coating composition is porous.

15. The stent of claim 11, wherein at least one of the first or second material comprises silica, melamine resin, polymethacrylate, polystyrene, polylactide, alumina, or a combination thereof.

16. The stent of claim 11, wherein said first material and said second material are bioabsorbable, and wherein said bonded first therapeutic agent is released from the stent after the first material is at least partially absorbed, and wherein said bonded second therapeutic agent is released from the stent after the second material is at least partially absorbed.

17. The stent of claim 11, wherein the average diameter of the molecule(s) of the first material is greater than the average diameter of the molecule(s) of the second material.

18. The stent of claim 11, wherein at least one of the first or second therapeutic agent comprises an anti-proliferative agent, anti-thrombogenic agent, anti-inflammatory agent, or a combination thereof.

19. The stent of claim 11, wherein at least one of the first or second therapeutic agent comprises rapamycin, daunomycin, mitocycin, dexamethasone, paclitaxel, everolimus, tacrolimus, zotarolimus, heparin, aspirin, warfarin, ticlopidine, salsalate, diflunisal, ibuprofen, ketoprofen, nabumetone, prioxicam, naproxen, diclofenac, indomethacin, sulindac, tolmetin, etodolac, ketorolac, oxaprozin, celcoxib, or a combination thereof.

20. The stent of claim 11, further comprising;
a polymeric coating composition disposed on at least a portion of the coating composition,
wherein said polymeric coating composition comprises a biostable, non-thrombogenic polymeric material, and wherein when the stent is implanted into a blood vessel, the bonded first therapeutic agent is released from the stent at a third rate that is different from the first rate, and the bonded second therapeutic agent is released from the stent at a fourth rate that is different from the second rate.

21. An intravascular stent comprising:
a substrate and a coating composition disposed on at least a portion of the substrate,
wherein the substrate comprises a first plurality of a first therapeutic agent and a second therapeutic agent;
wherein the coating composition comprise a second plurality of the first therapeutic agent and the second therapeutic agent;
wherein the first therapeutic agent is bonded to one or more molecule(s) of a first material to form a bonded first therapeutic agent, and the second therapeutic agent is bonded to one or more molecule(s) of a second material to form a bonded second therapeutic agent;
wherein the first therapeutic agent comprises an anti-proliferative agent, anti-thrombogenic agent, anti-inflammatory agent, or a combination thereof;
wherein said bonded first therapeutic agent is greater in size than said bonded second therapeutic agent; and
wherein when the stent is implanted into a blood vessel, the bonded first therapeutic agent is released from the stent at a first rate and the bonded second therapeutic agent is released from the stent at a second rate that is faster than said first rate.

22. The stent of claim 21, wherein said substrate comprises stainless steel, alumina film, platinum, cobalt, chromium, nickel, titanium, magnesium, or a combination thereof.

23. The stent of claim 21, wherein said substrate comprises polyethylene, polystyrene, polylactide, or a combination thereof.

24. The stent of claim 21, wherein at least one of the substrate or coating composition is porous.

25. The stent of claim 21, wherein at least one of the first or second material comprises silica, melamine resin, polymethacrylate, polystyrene, polylactide, alumina, or a combination thereof.

26. The stent of claim 21, wherein said first material and said second material are bioabsorbable, and wherein said bonded first therapeutic agent is released from the stent after the first material is at least partially absorbed, and wherein said bonded second therapeutic agent is released from the stent after the second material is at least partially absorbed.

27. The stent of claim 21, wherein the average diameter of the molecule(s) of the first material is greater than the average diameter of the molecule(s) of the second material.

28. The stent of claim 21, wherein at least one of the first or second therapeutic agent comprises an anti-proliferative agent, anti-thrombogenic agent, anti-inflammatory agent, or a combination thereof.

29. The stent of claim 21, wherein at least one of the first or second therapeutic agent comprises rapamycin, daunomycin, mitocycin, dexamethasone, paclitaxel, everolimus, tacrolimus, zotarolimus, heparin, aspirin, warfarin, ticlopidine, salsalate, diflunisal, ibuprofen, ketoprofen, nabumetone, prioxicam, naproxen, diclofenac, indomethacin, sulindac, tolmetin, etodolac, ketorolac, oxaprozin, celcoxib, or a combination thereof.

30. The stent of claim 21, further comprising:
a polymeric coating composition disposed on at least a portion of the coating composition,
wherein said polymeric coating composition comprises a biostable, non-thrombogenic polymeric material, and wherein when the stent is implanted into a blood vessel, the bonded first therapeutic agent is released from the stent at a third rate that is different from the first rate, and the bonded second therapeutic agent is released from the stent at a fourth rate that is different from the second rate.
